(19) United States
(12) Patent Application Publication
(10) Pub. No.: US 2002/0055488 A1
(43) Pub. Date: May 9, 2002

(54) Prevention and treatment of streptococcal and staphylococcal infection

(76) Inventors: Michael R. Wessels, Brookline, MA (US); Colette Cywes, Brookline, MA (US)

Correspondence Address: Edward R. Gates, Esq., Wolf, Greenfield & Sacks, P.C., 600 Atlantic Avenue, Boston, MA, 02210, US

(21) Appl. No.: 09/960,621

(22) Filed: Sep. 21, 2001

Related U.S. Application Data
(60) Provisional application No. 60/234,145, filed on Sep. 21, 2000.

(51) Int. Cl. A61K 31/728

(52) U.S. Cl. 514/54

(57) ABSTRACT

The invention provides new methods for use in prevention and treatment of streptococcal and staphylococcal infection.

### RELATED APPLICATIONS

This application claims priority under 35 U.S.C. §119 from U.S. provisional application Ser. No. 60/234,145, filed Sep. 21, 2000.

### GOVERNMENT SUPPORT

This invention was made in part with government support under grant number A129952 from the National Institutes of Health (NIH). The government may have certain rights in this invention.

### FIELD OF THE INVENTION

The invention relates to the prevention and treatment of streptococcal and staphylococcal infection.

### BACKGROUND OF THE INVENTION

Group A streptococcus (also known as Streptococcus pyogenes or GAS) and Group C streptococcus (GCS) are common human pathogens. Group A streptococcus is believed to colonize the pharynx leading directly to clinical manifestations such as streptococcal pharyngitis (strep throat), and indirectly to infections at other anatomic sites such as cellulitis or necrotizing fasciitis (hemolytic streptococcal gangrene); or to systemic infections such as streptococcal toxic shock syndrome, scarlet fever, sepsis, and bacteremia. Indirectly, GAS may also lead to post-infectious syndromes of acute rheumatic fever or glomerulonephritis. Group C streptococci, which may also colonize the pharynx, may result in human infections similar to those caused by Group A streptococci, including pharyngitis, pneumonia, cellulitis and soft-tissue infection, bacteremia, septic arthritis, and endocarditis. Group C bacteria may play a role in the aforementioned types of infections in the elderly or chronically ill patients; and in addition to its role as a human pathogen, Group C streptococcus is also a major pathogen in animals such as horses.

The onset of a disease, such as one of the aforementioned illnesses, is a complex event that results from factors including bacterial colonization, invasion, toxin elaboration, and the host's responses to these factors. One element in colonization is bacterial adhesion to a mucosal surface (Harrison's Principles of Internal Medicine, 1998). The pharynx, which is a mucosal surface, is believed to be a “reservoir” for GAS and GCS colonization and concomitant disease. In addition, to symptomatic infection, streptococcal infections are commonly asymptomatic, which means that although not clinically ill, the subject has pharyngeal colonization and is a carrier of the infection and capable of spreading the infection to others. Pharyngeal colonization may result in secondary infections and disease, as described herein. For example, even in the absence of clinical symptoms, bacteria asymptomatically colonizing the pharynx may be transferred from the throat to other regions of the body, resulting in infection. Therefore, treating GAS and GCS infections may comprise treating symptomatic disease and/or may comprise treating asymptomatic colonization.

CD44 has been identified as a receptor for Group A streptococcus. A monoclonal antibody to CD44 (IM7.8.1) has been shown in vitro to prevent binding, also known as adhesion, of Group A streptococcal bacteria (five types) to the receptor. Streptococcal bacteria are believed to attach to CD44 via the bacterial capsule, which is composed of hyaluronic acid. Although these findings serve to elucidate possible mechanisms of streptococcal binding in vitro, these results do not serve as a model for in vivo binding because significant differences between in vitro and in vivo environments exist. For example, the in vivo pharyngeal tissue, unlike the tissue in culture, is coated with a mucous layer, which makes hyaluronic acid access to CD44 on the pharyngeal cells unpredictable. In addition, presence of a wide assortment of cell and receptor types in the in vivo pharyngeal environment presents numerous obstacles to hyaluronic acid binding in vivo that are unparalleled in the simple binding parameters at work in the in vitro studies. These types of differences between the in vitro and in vivo environments render the findings from in vitro experiments not predictive of functional interference with disease and not suggestive of viable methods for in vivo applications. In short, the in vitro assays are not accepted models for in vivo binding behavior and efficacy.

Hyaluronic acid in the capsule is a simple linear polymer of β(1→4)-linked repeating units of β-D-glucuronic acid (→3) β-D-N-acetylglucosamine, and is structurally identical to mammalian hyaluronic acid. Hyaluronic acid has been utilized extensively in the cosmetic and pharmaceutical industry for numerous applications such as: lubrication of prosthetics, replacement fluids in eye surgery, an anti-inflammatory in arthritic joints, an anti-cancer agent, to prevent cavities, and as a carrier to allow other pharmaceuticals to access desirable sites of action in the body, but its administration, according to the invention, to reduce the likelihood of streptococcal infections, such as GAS and GCS, and staphylococcal infections is novel and unexpected.

### SUMMARY OF THE INVENTION

The invention involves the surprising discovery that agents that bind to the hyaluronic acid binding region of CD44 can interfere in vivo with adhesion, colonization, and disease caused by streptococcal bacteria.

According to one aspect of the invention, a method is provided for treating a subject to reduce the likelihood of streptococcal infection. The method involves administering orally to a subject in need of such treatment an agent that binds to a hyaluronic acid-binding region of a CD44 protein in an amount effective to interfere with adhesion of streptococcal bacteria to CD44 protein in the subject and inhibit streptococcal colonization of the pharynx. In one embodiment, the effective amount is that amount which statistically establishes a reduction in the likelihood of infection. In an important embodiment, the agent is administered to a subject suspected of exposure to Group A streptococcus or Group C streptococcus. In one embodiment, the subject is known to have been exposed to streptococcal bacteria. In another embodiment, the subject is known to be likely to be exposed to streptococcal bacteria. In another embodiment, the subject has been determined definitively to have been infected by streptococcal bacteria, and in yet another embodiment, the subject has not been determined to definitively to have been infected by streptococcal bacteria. In yet another embodiment, the subject is in need of such treatment to reduce the likelihood of streptococcal pharyngitis. In yet another embodiment, the CD44 protein in the subject in need of such treatment is located on the pharynx. Preferred agents are hyaluronic acid, analogs of hyaluronic acid and peptides, including antibodies.

According to another aspect of the invention, a method is provided for treating a subject to reduce the likelihood of streptococcal infection. The method involves administering orally to a subject in need of such treatment an agent that binds to a hyaluronic acid-binding region of the CD44 protein in an amount effective to displace streptococcal bacteria bound to CD44 protein in the subject and inhibit streptococcal colonization of the pharynx. In one embodiment, the effective amount is that amount which statistically reduces the likelihood of infection in the subject.

The subject may have been determined to have a streptococcal infection or the subject may not have been determined to have a streptococcal infection. In one embodiment, the agent is administered to a subject suspected of having been exposed to, or having a future risk of exposure to, Group A streptococcus or Group C streptococcus. Preferably, the subject is in need of the treatment to reduce the likelihood of streptococcal pharyngitis. Also, preferably the CD44 protein in the subject is located in the pharynx. Most preferably the subject is a human.

The agent can be any agent that binds to a hyaluronic acid-binding region of a CD44 protein as defined and described below. Preferably, the agent is hyaluronic acid, an analog of hyaluronic acid, or a peptide, for example, an antibody.

According to another aspect of the invention, a method is provided for treating a subject to reduce the likelihood of streptococcal or staphylococcal infection. The method involves administering orally to a subject in need of such treatment an agent that binds to a hyaluronic acid-binding region of a CD44 protein of a mucosal membrane in an amount effective to interfere with adhesion of streptococcal or staphylococcal bacteria to the mucosal membrane in the subject. In one embodiment, the hyaluronic acid interferes with adhesion of streptococcal bacteria to CD44 protein of a mucosal membrane in the subject. In certain embodiments, the effective amount of the agent administered to the subject in need of such treatment statistically reduces the likelihood of infection. In some embodiments, the agent is administered to a subject suspected of exposure to Group A streptococcus, Group C streptococcus, Group B streptococcus, Streptococcus pneumoniae, or Staphylococcus aureus. In certain embodiments, the subject is known to have been exposed to streptococcal or staphylococcal bacteria. In some embodiments, the subject is likely to be exposed to streptococcal or staphylococcal bacteria.

According to yet another aspect of the invention, a method is provided for treating a subject to reduce the likelihood of streptococcal or staphylococcal infection. The method involves administering orally to a subject in need of such treatment an agent that binds to a hyaluronic acid-binding region of a CD44 protein of a mucosal membrane in an amount effective to displace streptococcal or staphylococcal bacteria bound to the mucosal membrane in the subject. In some embodiments, the hyaluronic acid interferes with adhesion of streptococcal bacteria to CD44 protein of a mucosal membrane in the subject. In certain embodiments, the effective amount of the agent administered to the subject in need of such treatment statistically reduces the likelihood of infection. In some embodiments, the agent is administered to a subject suspected of exposure to Group A streptococcus, Group C Streptococcus, Group B streptococcus, Streptococcus pneumoniae, or Staphylococcus aureus. In some embodiments, the subject has not been determined to have streptococcal or staphylococcal infection. In other embodiments, the subject has been determined to have streptococcal or staphylococcal infection.

According to another aspect of the invention, a method is provided for treating a subject to reduce the likelihood of streptococcal or staphylococcal infection. The method involves administering nasally to a subject in need of such treatment who does not have a nasal cavity wound, an agent that binds to a hyaluronic acid-binding region of a CD44 protein of a mucosal membrane in an amount effective to interfere with adhesion of streptococcal or staphylococcal bacteria to the mucosal membrane in the subject. In some embodiments, the hyaluronic acid interferes with adhesion of streptococcal bacteria to CD44 protein of a mucosal membrane in the subject. In certain embodiments, the effective amount of the agent administered to the subject in need of such treatment statistically reduces the likelihood of infection. In some embodiments, the agent is administered to a subject suspected of exposure to Group A streptococcus, Group C streptococcus, Group B streptococcus, Streptococcus pneumoniae, or Staphylococcus aureus. In some embodiments, the subject is known to have been exposed to streptococcal or staphylococcal bacteria. In other embodiments, the subject is likely to be exposed to streptococcal or staphylococcal bacteria. In certain embodiments, the CD44 protein of the mucosal membrane in the subject in need of such treatment is on a nasal mucosal membrane.

According to yet another aspect of the invention, a method is provided for treating a subject to reduce the likelihood of streptococcal or staphylococcal infection. The method involves administering nasally to a subject in need of such treatment who does not have a nasal cavity wound, an agent that binds to a hyaluronic acid-binding region of a CD44 protein of a mucosal membrane in an amount effective to displace streptococcal or staphylococcal bacteria bound to the mucosal membrane in the subject. In some embodiments, the hyaluronic acid interferes with adhesion of streptococcal bacteria to CD44 protein of a mucosal membrane in the subject. In certain embodiments, the effective amount of the agent administered to the subject in need of such treatment statistically reduces the likelihood of infection. In some embodiments, the agent is administered to a subject suspected of exposure to Group A streptococcus, Group C streptococcus, Group B streptococcus, Streptococcus pneumoniae, or Staphylococcus aureus. In certain embodiments, the subject has not been determined to have streptococcal or staphylococcal infection. In other embodiments, the subject has been determined to have streptococcal or staphylococcal infection. In some embodiments, the CD44 protein of the mucosal membrane in the subject in need of such treatment is on a nasal mucosal membrane.

In some embodiments of the foregoing methods, the subject is a human. In some embodiments of the foregoing methods, the subject is in need of such treatment to reduce the likelihood of streptococcal or staphylococcal pharyngitis. In certain embodiments of the foregoing methods, the CD44 protein of the mucosal membrane in the subject in need of such treatment is on the pharynx. In some embodiments of the foregoing methods, the agent is hyaluronic acid or an analog of hyaluronic acid. In other embodiments of the foregoing methods, the agent is a peptide and in certain embodiments of the foregoing methods, the peptide is an antibody.

According to another aspect of the invention, a medicinal product is provided. The medicinal product is a syrup containing an amount of hyaluronic acid effective to interfere with adhesion of streptococcal bacteria to CD44 protein in a subject and to inhibit streptococcal colonization of a pharynx of the subject.

According to another aspect of the invention, a medicinal product is provided. The medicinal product is a syrup containing an amount of hyaluronic acid effective to displace streptococcal bacteria bound to CD44 protein in a subject and to inhibit streptococcal colonization of a pharynx of the subject.

In some embodiments of the foregoing products, the streptococcal bacteria is Group A streptococcal bacteria, and in some embodiments the streptococcal bacteria is Group C streptococcal bacteria. In some embodiments of the foregoing products, the subject is known to have been exposed to streptococcal bacteria. In other embodiments of the foregoing products, the subject is likely to be exposed to streptococcal bacteria. In some embodiments of the foregoing products, the subject is a human. In some embodiments of the foregoing products, the subject is in need of such treatment to reduce the likelihood of streptococcal pharyngitis. In certain embodiments of the foregoing products, the CD44 protein of the mucosal membrane in the subject in need of such treatment is on a pharynx of the subject.

According to another aspect of the invention, a medicinal product is provided. The medicinal product is a syrup containing an amount of hyaluronic acid effective to interfere with adhesion of streptococcal or staphylococcal bacterial to a mucosal membrane in a subject. In some embodiments, the mucosal membrane in the subject is a pharynx of the subject.

According to another aspect of the invention, a medicinal product is provided. The medicinal product is a syrup containing an amount of hyaluronic acid effective to displace streptococcal or staphylococcal bacteria bound to a mucosal membrane in a subject. In some embodiments, the mucosal membrane in the subject is a pharynx of the subject.

In some embodiments of the foregoing products, the bacteria is Group A streptococcus, Group C streptococcus, Group B streptococcus, Streptococcus pneumoniae, or Staphylococcus aureus. In some embodiments of the foregoing methods, the subject is a human. In some embodiments of the foregoing methods, the subject is in need of such treatment to reduce the likelihood of streptococcal or staphylococcal pharyngitis. In certain embodiments of the foregoing methods, the CD44 protein of the mucosal membrane in the subject in need of such treatment is on a pharynx of the subject.

In some embodiments of the foregoing methods, the preferred syrup is a cough syrup as described in greater detail below in the Detailed Description. The cough syrup can further contain an expectorant, decongestant, cough suppressant, and/or pain reliever/fever reducer.

According to another aspect of the invention, a medicinal product is provided. The medicinal product is a frozen solution containing an amount of hyaluronic acid effective to interfere with adhesion of streptococcal bacteria to CD44 protein in a subject and to inhibit streptococcal colonization of a pharynx of the subject.

According to another aspect of the invention, a medicinal product is provided. The product is a frozen solution containing an amount of hyaluronic acid effective to displace streptococcal bacteria bound to CD44 protein in a subject and to inhibit streptococcal colonization of a pharynx of the subject.

In some embodiments of the foregoing products, the streptococcal bacteria is Group A streptococcal bacteria and in some embodiments the streptococcal bacteria is Group C streptococcal bacteria. In some embodiments of the foregoing products, the subject is known to have been exposed to streptococcal bacteria. In other embodiments of the foregoing products, the subject is likely to be exposed to streptococcal bacteria. In some embodiments of the foregoing products, the subject is a human. In some embodiments of the foregoing products, the subject is in need of such treatment to reduce the likelihood of streptococcal pharyngitis. In certain embodiments of the foregoing products, the CD44 protein of the mucosal membrane in the subject in need of such treatment is on a pharynx of the subject.

According to yet another aspect of the invention, a medicinal product is provided. The medicinal product is a frozen solution containing an amount of hyaluronic acid effective to interfere with adhesion of streptococcal or staphylococcal bacteria to a mucosal membrane in a subject. In some embodiments, the mucosal membrane in the subject is a pharynx of the subject.

According to another aspect of the invention, a medicinal product is provided. The medicinal product is a frozen solution containing an amount of hyaluronic acid effective to displace streptococcal or staphylococcal bacteria bound to a mucosal membrane in a subject. In preferred embodiments, the mucosal membrane in the subject is a pharynx of the subject.

In some embodiments of the foregoing products, the bacteria is Group A streptococcus, Group C streptococcus, Group B streptococcus, Streptococcus pneumoniae, or Staphylococcus aureus. In some embodiments of the foregoing methods, the subject is a human. In some embodiments of the foregoing methods, the subject is in need of such treatment to reduce the likelihood of streptococcal or staphylococcal pharyngitis. In certain embodiments of the foregoing methods, the CD44 protein of the mucosal membrane in the subject in need of such treatment is on a pharynx of the subject.

The frozen solution medicinal product is described in greater detail below in the Detailed Description.

According to another aspect of the invention, a medicinal product is provided. The medicinal product is a solid solution containing an amount of hyaluronic acid effective to interfere with adhesion of streptococcal bacteria to CD44 protein in a subject and to inhibit streptococcal colonization of a pharynx in a subject.

According to another aspect of the invention, a medicinal product is provided. The medicinal product is a solid solution containing an amount of hyaluronic acid effective to displace streptococcal bacteria bound to CD44 protein in a subject and to inhibit streptococcal colonization of a pharynx of the subject.

In some embodiments of the foregoing products, the streptococcal bacteria is Group A streptococcal bacteria and in some embodiments the streptococcal bacteria is Group C streptococcal bacteria. In some embodiments of the foregoing products, the subject is known to have been exposed to streptococcal bacteria. In other embodiments of the foregoing products, the subject is likely to be exposed to streptococcal bacteria. In some embodiments of the foregoing products, the subject is a human. In some embodiments of the foregoing products, the subject is in need of such treatment to reduce the likelihood of streptococcal pharyngitis. In certain embodiments of the foregoing products, the CD44 protein of the mucosal membrane in the subject in need of such treatment is on a pharynx of the subject.

According to another aspect of the invention, a medicinal product is provided. The medicinal product is a solid solution containing an amount of hyaluronic acid effective to interfere with adhesion of streptococcal or staphylococcal bacteria to a mucosal membrane in a subject. In preferred embodiments, the mucosal membrane in the subject is a pharynx of the subject.

According to another aspect of the invention, a medicinal product is provided. The medicinal product is a solid solution containing an amount of hyaluronic acid effective to displace streptococcal or staphylococcal bacteria bound to a mucosal membrane in a subject. In preferred embodiments, the mucosal membrane in the subject is a pharynx of the subject.

In some embodiments of the foregoing products, the bacteria is Group A streptococcus, Group C streptococcus, Group B streptococcus, Streptococcus pneumoniae, or Staphylococcus aureus. In some embodiments of the foregoing methods, the subject is a human. In some embodiments of the foregoing methods, the subject is in need of such treatment to reduce the likelihood of streptococcal or staphylococcal pharyngitis. In certain embodiments of the foregoing methods, the CD44 protein of the mucosal membrane in the subject in need of such treatment is on a pharynx of the subject. The solid solution medicinal product is described in greater detail below in the Detailed Description.

These and other aspects of the invention will be described in greater detail below in connection with the drawings.

### BRIEF DESCRIPTION OF THE DRAWINGS

FIG. 1 is a graph illustrating the inhibition of GAS attachment to normal mouse keratinocytes by mAb to CD44. Data represent mean ±SD of adherent bacteria recovered after inoculation of keratinocytes with GAS in the absence (filled bars) or presence (hatched bars of anti-CD44 mAb KM81, 15 μg/ml. KM81-treatment inhibited attachment of wild-type (encapsulated) GAS strains B514-Sm and 950771, but not the acapsular mutant strain 188.

FIG. 2 is a graph demonstrating GAS attachment to wild-type or CD44-deficient mouse keratinocytes. Data represent mean ±SD of adherent bacteria recovered after inoculation of wild-type (solid bars) or K5-CD44 antisense (CD44-deficient, hatched bars) keratinocytes with wild-type (encapsulated) GAS strain B514-Sm or 950771 or with their respective acapsular mutants, UABO39 and 188. Attachment of the wild-type GAS strains, but not the acapsular mutant strains, to CD44-deficient keratinocytes was reduced by approximately 75% compared to attachment to wild-type keratinocytes.

FIG. 3 illustrates GAS pharyngeal colonization of wild-type mice and of mice deficient in expression of epithelial CD44. (A) is a photomicrographic image of histologic section through the pharynx of a representative wild-type mouse stained with mAb to CD44. Immunohistochemical staining of the pharyngeal epithelium is seen with mAb to CD44, but not with an irrelevant control mAb. Labels indicate the location of the epithelium (E), lumen (L), and submucosa (S). (B) is a table in which the left column shows the level of CD44 expression in the pharyngeal epithelium of wild-type and K5-CD44 antisense mice. Histologic sections were scored for CD44 expression in the pharyngeal epithelium by 3 independent observers without knowledge of the throat culture results. CD44 expression was graded from 1 (background) to 4 (equivalent to wild-type control). On the right are results of throat cultures for GAS on each of 5 days after intranasal inoculation with GAS B514-Sm. (C) is a histogram of summary of throat culture results presented in panel B. Data represent the percentage of mice with a positive throat culture on each day after intranasal inoculation for wild-type mice (solid bars), transgenic mice with wild-type levels of CD44 on keratinocytes (hatched bars), and transgenic mice with reduced or absent CD44 expression on keratinocytes (open bars).

FIG. 4 is two graphs that illustrate the prevention of GAS pharyngeal colonization in vivo by anti-CD44 monoclonal antibody. Data represent the fraction of mice with positive throat cultures for GAS on each of 3 days after intranasal inoculation with GAS administered either with mAb to CD44 or with an irrelevant control mAb (n=20 mice per group).

FIG. 5 is three graphs that demonstrate inhibition of GAS attachment to mouse keratinocytes by exogenous hyaluronic acid. Data represent mean ±SD of adherent bacteria recovered after incubation of keratinocytes with GAS in the presence of no inhibitor, hyaluronic acid (HA), or a control polysaccharide, alginic acid (AL), at the indicated times after addition of GAS to the keratinocytes.

FIG. 6 is three graphs that illustrate the prevention of GAS pharyngeal colonization by pretreatment with hyaluronic acid. Data represent the fraction of mice with positive throat cultures for GAS on each of 3 days after intranasal inoculation with GAS following pretreatment with phosphate-buffered saline, hyaluronic acid (HA), or a control polysaccharide, alginic acid (AL) (n=13 mice per group).

FIG. 7 is a graph showing the effect of hyaluronic acid added exogenously in preventing adhesion of pathogenic bacteria to human keratinocytes.

FIG. 8 is three drawings of which (A) is a syrup medicinal product (8) with syrup (12) in a container (10), (B) is a solid solution medicinal product (14), with a solid solution (16), and a handle (18), and (C) is a frozen solution medicinal product (20), with a frozen solution (22), and a handle (24).

### DETAILED DESCRIPTION OF THE INVENTION

The invention is based on the surprising finding that the oral administration of hyaluronic acid results in hyaluronic acid binding to CD44 and blocking the adhesion and colonization of streptococcal and staphylococcal bacteria such as Group A streptococcus, Group B streptococcus, Group C streptococcus, streptococcus pneumoniae, and staphylococcus aureus in the pharynx and nasal mucosa. Accordingly, the invention is related to new uses for hyaluronic acid in subjects, in view of its newly discovered function, namely a role in in vivo reduction in streptococcal and staphylococcal binding and colonization in the pharynx, a known reservoir for streptococcal infection. The invention also relates to the use of other agents that bind to the hyaluronic acid binding region of CD44, such as nonpeptide organic agents and peptides, for in vivo reduction of streptococcal and staphylococcal adhesion and colonization of the pharynx and to the use of the agents to reduce the likelihood of Group A streptococcus, Group B streptococcus, Group C streptococcus, streptococcus pneumoniae, and staphylococcus aureus binding and infection.

As used herein, a subject is a human, non-human primate, cow, horse, pig, sheep, goat, dog, cat, or rodent. In all embodiments human subjects are preferred. Particularly important subjects to which the present invention can be applied are subjects diagnosed as having been or suspected of having been exposed to Group A streptococcus, Group B streptococcus, Group C streptococcus, streptococcus pneumoniae, and staphylococcus aureus, which includes subjects diagnosed with infection, exhibiting symptoms of infection, or having known or probable risk of exposure to an individual known to have streptococcal or staphylococcal infection. For example, close family members of an individual testing positive for streptococcal or staphylococcal infection would be suspected of having been exposed to or at risk of future exposure to streptococcal or staphylococcal bacteria.

A subject may or may not exhibit symptoms of infection such as streptococcal pharyngitis, which is also known as strep throat. If present, symptoms may include sore throat, fever, chills, vomiting, swelling of the pharyngeal mucosa, and purulent exudate over the posterior pharyngeal wall and tonsillar pillars (Harrison's Principles of Internal Medicine, 1998). Methods to diagnose symptomatic and asymptomatic streptococcal or staphylococcal infection are known to those of ordinary skill in the medical arts and include, but are not limited to, swab of affected region for bacterial culture or rapid streptococcal diagnostic testing such as the latex agglutination or enzyme immunoassay of swab specimens. Infected regions may include the throat, nasal mucosa, or other body regions. In an important embodiment of the invention the infection is in the pharynx.

Treatment as it relates to the invention may be prophylactic or post-infection. Prophylactic treatment may comprise administering hyaluronic acid, a hyaluronic acid analog, or other agent that binds the hyaluronic acid binding region of CD44, to interfere with streptococcal binding, which is also known as adhesion, to CD44 protein in the subject. Post-exposure or post-infection treatment may comprise administering hyaluronic acid, a hyaluronic acid analog, or other agent that binds to the hyaluronic acid binding region of CD44 to displace streptococcal adhesion to CD44 protein in the subject. Thus, in an important embodiment, the hyaluronic acid, analog, or other agent that binds to the hyaluronic acid binding region of CD44 is administered to inhibit colonization (i.e. further binding) or even to displace streptococcal binding and/or to interfere with streptococcal binding in a subject. As used herein, the term “inhibit colonization” means to reduce or lower the level of colonization. To inhibit may also mean to prevent colonization, but it is not necessary to prevent all colonization to lessen or prevent the manifestation of disease.

Agents can be prepared that bind specifically to CD44 and/or the hyaluronic acid binding region of CD44. As used herein, “binding specifically to the hyaluronic acid binding region of CD44” means binding to and distinguishing the hyaluronic acid binding region of CD44 from other regions of CD44. As used herein, “binding specifically to CD44” means binding to and distinguishing CD44 from other naturally occurring proteins. Agents that bind to CD44 molecules, including but not limited to the hyaluronic acid binding region of CD44 molecules, include hyaluronic acid, also known as sodium hyaluronate, analogs thereof, and organic agents. Polypeptide agents include polyclonal and monoclonal antibodies, prepared according to conventional methodology. Certain antibodies useful in the methods of the invention already are known in the art and include certain anti-CD44 antibodies, for example the monoclonal antibody IM7.8.1, which binds to CD44.

An “agent that binds to the hyaluronic acid binding region of CD44” is one that binds to the hyaluronic acid-binding site on CD44 and interferes with streptococcal binding to the site, or binds close enough to the hyaluronic binding site to interfere with streptococcal binding. Such agents include nonpeptide and peptide agents. Such agents can be organic agents identified, for example, using routine combinatorial screening methodology or through immunological procedures. Since the binding site and its ligand are known, rationally based combinatorial libraries can be prepared, based on computer-generated models of the binding site and/or 3-dimensional structural information obtained from crystals of the bound complex.

Such agents also can be synthesized chemically from peptides or other biomolecules including but not limited to saccharides, fatty acids, sterols, isoprenoids, purines, pyrimidines, derivatives or structural analogs of the above, or combinations thereof and the like. Phage display libraries and chemical combinatorial libraries can be used to develop and select synthetic compounds which bind to the hyaluronic acid binding region of CD44. Also envisioned in the invention is the use of agents made from peptoids, random bio-oligomers (U.S. Pat. No. 5,650,489), benzodiazepines, diversomeres such as dydantoins, benzodiazepines and dipeptides, nonpeptidal peptidomimetics with a beta-D-glucose scaffolding, oligocarbamates or peptidyl phosphonates.

The agents of the invention thus may be produced en masse using library technology. In some aspects, the methods of the invention utilize this library technology to generate and subsequently identify small molecules, including small peptides, that bind to the hyaluronic acid binding region of CD44 molecules. One advantage of using libraries is the facile manipulation of millions of different putative candidates of small size in small reaction volumes (i.e., in synthesis and screening reactions). Another advantage of libraries is the ability to synthesize agents which might not otherwise be attainable using naturally occurring sources, particularly in the case of non-peptide moieties. A “molecular library” refers to a collection of structurally-diverse molecules. Molecular libraries can be chemically-synthesized or recombinantly produced. As used herein, a “molecular library member” refers to a molecule that is present within the molecular library. In general, a molecular library contains from two to 10¹² molecules, and any integer number therebetween, e.g., 2, 3, 4, 5, 10, 10², 10³, 10⁴, 10⁵, 10⁶, 10⁷, 10⁸, 10⁹, 10¹⁰, 10¹¹, 10¹¹, 10¹² and so forth, as if each and every integer has been recited herein.

Methods for preparing libraries of molecules are well known in the art and many libraries are commercially available. Libraries of interest in the invention include peptide libraries, randomized oligonucleotide libraries, synthetic organic combinatorial libraries, and the like. Degenerate peptide libraries can be readily prepared in solution, in immobilized form as bacterial flagella peptide display libraries or as phage display libraries. Peptide ligands can be selected from combinatorial libraries of peptides containing at least one amino acid. Libraries can be synthesized of peptoids and non-peptide synthetic moieties. Such libraries can further be synthesized which contain non-peptide synthetic moieties which are less subject to enzymatic degradation compared to their naturally-occurring counterparts. Libraries are also meant to include for example but are not limited to peptide or plasmid libraries, polysome libraries, aptamer libraries, synthetic peptide libraries, synthetic small molecule libraries and chemical libraries. The libraries can also comprise cyclic carbon or heterocyclic structure and/or aromatic or polyaromatic structures substituted with one or more of the above-identified functional groups.

Many, if not all, of these agents of the invention can be synthesized using recombinant or chemical library approaches. A vast array of candidate agonists or antagonists can be generated from libraries of synthetic or natural compounds. Libraries of natural compounds in the form of bacterial, fungal, plant and animal extracts are available or can readily produced. Natural and synthetically produced libraries and compounds can be readily modified through conventional chemical, physical, and biochemical means. Known binding partners of CD44 molecules may be subjected to directed or random chemical modifications such as acylation, alkylation, esterification, amidification, etc. to produce structural analogs of these binding partners. Synthetic DNA and RNA libraries are also commonly used in the art. For instance, Ellington and Szostak describe the use of random polynucleotide libraries to identify novel ligands [Ellington and Szostak, Nature, 346, 818-822 (1990)].

As described in U.S. Pat. No. 5,908,609, exemplary library compounds also include, but are not limited to, peptides such as, for example, soluble peptides, including but not limited to members of random peptide libraries; (see, e.g., Lam, K. S. et al. 1991, Nature 354:82-84; Houghten, R. et al. 1991, Nature 354:84-86), and combinatorial chemistry-derived molecular libraries made of D- and/or L-configuration amino acids, phosphopeptides (including but not limited to, members of random or partially degenerate, directed phosphopeptide libraries; (see, e.g., Songyang, Z. et al. 1993, Cell 72: 767-778), antibodies (including, but not limited to, polyclonal, monoclonal, humanized, anti-idiotypic, chimeric or single chain antibodies, and FAb, F(ab′)₂ and FAb expression library fragments (and epitope-binding fragments thereof), and small organic or inorganic molecules. Compounds that can be designed to satisfy the foregoing criteria include polypeptides and peptide mimetics. The peptide mimetic can be a hybrid molecule which includes both amino acid and non-amino acid components, e.g., the mimic can include amino acid components for the positively charged and negatively charged regions and a non-amino acid (e.g., piperidine) having the same approximate size and dimension of a hydrophobic amino acid (e.g., phenylalanine) as the hydrophobic component.

Small molecule combinatorial libraries may also be generated. A combinatorial library of small organic compounds is a collection of closely related analogs that differ from each other in one or more points of diversity and are synthesized by organic techniques using multi-step processes. Combinatorial libraries include a vast number of small organic compounds. One type of combinatorial library is prepared by means of parallel synthesis methods to produce a compound array. A “compound array” as used herein is a collection of compounds identifiable by their spatial addresses in Cartesian coordinates and arranged such that each compound has a common molecular core and one or more variable structural diversity elements. The compounds in such a compound array are produced in parallel in separate reaction vessels, with each compound identified and tracked by its spatial address. Examples of parallel synthesis mixtures and parallel synthesis methods are provided in U.S. Ser. No. 08/177,497, filed Jan. 5, 1994 and its corresponding PCT published patent application W095/18972, published Jul. 13, 1995 and U.S. Pat. No. 5,712,171 granted Jan. 27, 1998 and its corresponding PCT published patent application W096/22529, which are hereby incorporated by reference.

Thus, according to still another aspect of the invention, low-molecular-weight compounds that inhibit the interaction between hyaluronic acid and a CD44 molecule are provided. These compounds can be used to modulate the interaction or can be used as lead compounds for the design of better compounds using the computer-based rational drug design methods.

Other methods for preparing or identifying peptides which bind to a particular target are known in the art. Molecular imprinting, for instance, may be used for the de novo construction of macromolecular structures such as peptides which bind to a particular molecule. See, for example, Kenneth J. Shea, Molecular Imprinting of Synthetic Network Polymers: The De Novo synthesis of Macromolecular Binding and Catalytic Sites, TRIP Vol. 2, No. 5, May 1994; Klaus Mosbach, Molecular Imprinting, Trends in Biochem. Sci., 19(9) January 1994; and Wulff, G., in Polymeric Reagents and Catalysts (Ford, W. T., Ed.) ACS Symposium Series No. 308, pp 186-230, American Chemical Society (1986). As an example, one method for preparing mimics of hyaluronic acid involves the steps of: (i) polymerization of functional monomers around a known substrate (the template or in this case, the hyaluronic acid- binding region) that exhibits a desired activity; (ii) removal of the template molecule; and then (iii) polymerization of a second class of monomers in the void left by the template, to provide a new molecule which exhibits one or more desired properties which are similar to that of the template. In addition to preparing peptides in this manner, other binding molecules such as polysaccharides, nucleosides, drugs, nucleoproteins, lipoproteins, carbohydrates, glycoproteins, steroids, lipids, and other biologically active materials can also be prepared. This method is useful for designing a wide variety of biological mimics that are more stable than their natural counterparts, because they are typically prepared by the free radical polymerization of functional monomers, resulting in a compound with a nonbiodegradable backbone. Other methods for designing such molecules include, for example, drug design based on structure activity relationships which require the synthesis and evaluation of a number of compounds and molecular modeling.

In yet another approach to the identification of hyaluronic acid mimics, antibodies which bind to the CD44 molecule are also embraced by the invention. Antibodies include polyclonal and monoclonal antibodies, prepared according to conventional methodology. Antibodies to the CD44 molecule can be generated using techniques standard in the art. Antibodies generated in such a manner can then be manipulated in a number of ways, as described below.

Another category relevant to interference with binding of hyaluronic acid and CD44 is peptides. Significantly, as is well-known in the art, only a small portion of an antibody molecule, the paratope, is involved in the binding of the antibody to its epitope (see, in general, Clark, W. R. (1986) The Experimental Foundations of Modem Immunology, Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (Frs), which maintain the tertiary structure of the paratope (see, in general, Clark, W. R. (1986) The Experimental Foundations of Modem Immunology, Wiley & Sons, Inc., New York; Roitt, I. (1991) Essential Immunology, 7th Ed., Blackwell Scientific Publications, Oxford). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. See, e.g., U.S. Pat. Nos. 4,816,567, 5,225,539, 5,585,089, 5,693,762 and 5,859,205.

Thus, for example, PCT International Publication Number WO 92/04381 teaches the production and use of humanized murine RSV antibodies in which at least a portion of the murine FR regions have been replaced by FR regions of human origin. Such antibodies, including fragments of intact antibodies with antigen-binding ability, are often referred to as “chimeric” antibodies.

Thus, as will be apparent to one of ordinary skill in the art, the present invention also provides for F(ab′)₂, Fab, Fv and Fd fragments; chimeric antibodies in which the Fc and/or Fr and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or nonhuman sequences. The present invention also includes so-called single chain antibodies.

Thus, the invention involves polypeptides of numerous size and type that bind specifically to the hyaluronic acid binding region of CD44 molecules and polypeptides that bind close enough to the hyaluronic acid bind region of CD44 to interfere with hyaluronic acid binding. These polypeptides may be derived also from sources other than antibody technology, as described above.

As mentioned above, peptides which bind to the hyaluronic acid or the CD44 molecule can be generated and identified by conventional screening methods such as phage display procedures (e.g., methods described in Hart, et al. J. Biol. Chem. 269:12468 (1994)). Hart et al. report a filamentous phage display library for identifying novel peptide ligands for mammalian cell receptors. In general, phage display libraries using, e.g., M13 or fd phage, are prepared using conventional procedures such as those described in the foregoing reference. The libraries display inserts containing from 4 to 80 amino acid residues. The inserts optionally represent a completely degenerate or a biased array of peptides. Ligands that bind selectively to a receptor site are obtained by selecting those phages which express on their surface an amino acid sequence which recognizes and binds to the receptor. As used in this example, the CD44 molecule can be used as a receptor and the ligands are the peptides produced and displayed by the phage. These phage then are subjected to several cycles of reselection to identify those which have the most useful binding characteristics. The minimal linear portion of the sequence that binds to the ligand binding site can be determined. Typically, phage that exhibit the best binding characteristics (e.g., highest affinity) are further characterized by nucleic acid analysis to identify the particular amino acid sequences of the peptides expressed on the phage surface and the optimum length of the expressed peptide to achieve optimum binding.

One can repeat the procedure using a biased library containing inserts containing part of all of the minimal linear portion plus one or more additional degenerate residues upstream or downstream thereof. Yeast two-hybrid screening methods also may be used to identify polypeptides that bind to the CD44 molecules or hyaluronic acid binding region of CD44 molecules. Thus, CD44 molecules, or fragments thereof containing the hyaluronic acid binding region of CD44, can be used to screen peptide libraries, including phage display libraries, to identify and select peptide binding partners of the CD44 or hyaluronic acid binding region of CD44 molecules.

The displayed peptide sequence can vary in size. As the size increases, the complexity of the library increases. It is preferred that the total size of the displayed peptide sequence (the random amino acids plus any spacer amino acids) should not be greater than about 100 amino acids long, more preferably not greater than about 50 amino acids long, and most preferably not greater than about 25 amino acids long.

In certain embodiments, the libraries may have at least one constraint imposed upon the displayed peptide sequence. A constraint includes, but is not limited to, a positive or negative charge, hydrophobicity, hydrophilicity, a cleavable bond and the necessary residues surrounding that bond, and combinations thereof. In certain embodiments, more than one constraint is present in each of the peptide sequences of the library.

As mentioned above, the invention further embraces peptidomimetic residues, including non-naturally occurring amino acids. Such variants can be synthesized by substituting amino acids residues involved in the interaction between the peptide agent and the CD44 molecule with peptidomimetic residues. For example, glutamine (Glu) residues may be replaced with α-aminoadipate molecules and tyrosine positions may be substituted with 4-carboxymethyl-Phe. Phosphorus and non-phosphorus based analogs, such as phosphorotyrosine mimetics, may be used in the variants. Tyrosine analogs which can be used in place of the tyrosine residues include phenylalanine (Phe), pentafluoro phenylalanine (PfPhe), 4-carboxymethyl-L-phenylalanine (cmPhe), 4-carboxydifluoromethyl-L-phenylalanine (F₂ cmPhe), 4-phosphonomethyl-phenylalanine (Pmp), (difluorophosphonomethyl)phenylalanine (F₂Pmp), O-malonyl-L-tyrosine (malTyr or OMT), and fluoro-O-malonyltyrosine (FOMT). Phosphonate-based mimetics which substitute a methylene unit for the tyrosyl phosphate ester bond may also be incorporated into synthetic agonists and antagonists. Additionally, glutamic acid residues can be modified to possess an additional methylene group or they may simply be substituted with α-amino-adipate (Adi). Other residues which may be used include the non-naturally occurring amino acid 1-aminocyclohexylcarboxylic acid (Ac₆c) and 3-(2-hydroxynaphtalen-1-yl)-propyl, or 2-azetidinecarboxylic acid or pipecolic acid (which have 6-membered, and 4-membered ring structures respectively) for proline residues, S-ethylisothiourea, 2-NH₂-thiazoline and 2-NH₂-thiazole. Also useful in the synthesis of variants is the use of asparagine residue substitutes such as 3-indolyl-propyl. It will be apparent to one of ordinary skill in the art that the invention embraces the synthesis of a wide variety of variants having any combination of amino acid analogs and/or peptidomimetic residues as described above and as are known in the art. Further potential modifications envisioned by the invention include modifications of cysteines, histidines, lysines, arginines, tyrosines, glutamines, asparagines, prolines, and carboxyl groups are well known in the art and are described in U.S. Pat. No. 6,037,134. Synthesis of the aforementioned variants is described in the cited references and is well within the realm of one of ordinary skill in the art.

The variants may also be modified to introduce or stabilize certain structural features. As an example, β-bends may be incorporated into the, preferably peptide, variants or the variants may be synthesized as cyclic peptides for example by incorporating thio-ether linkages.

The foregoing antibodies and other binding molecules may be used for example to isolate and identify CD44 protein and/or the hyaluronic acid binding region of CD44 protein. The antibodies may be coupled to specific diagnostic labeling agents for imaging of the protein or fragment thereof. The antibodies may also be used for immunoprecipitation, immunoblotting CD44 and/or hyaluronic acid binding region of CD44 molecules and/or in competitive binding assays using standard methods known to those of ordinary skill in the art.

Once a putative agent had been identified, it then can be tested in vitro in competitive binding assays with killed or live bacteria, and then in animals and humans, as described in the examples before.

An “effective amount” of a treatment is that amount of hyaluronic acid, analog, or other agent that binds to the hyaluronic acid binding region of CD44 that alone, or together with further doses, produces the desired response, e.g. interference with Group A or Group C streptococcal binding to CD44 protein in the subject. In the case of treating a particular disease or condition, the desired response is inhibiting the onset or progression of the disease or condition, for example streptococcal pharyngitis. This may involve reducing but not necessarily preventing or eliminating the colonization of streptococcal or staphylococcal bacteria, although more preferably, it involves preventing or eliminating colonization of streptococcal or staphylococcal bacteria in the subject. The subject's response to treatment also may be a delay in the onset of the disease or condition.

The subject's response to treatment may be determined either directly or indirectly. Direct methods include, but are not limited to, standard swabbing and culturing, or rapid detection methods as described herein. Indirect methods of determining a subject's response may utilize statistical analysis based on the therapeutic responses to a plurality of dose and administration regimens administered to a plurality of subjects. Indirect methods may also include assessment of changes in symptoms of streptococcal or staphylococcal infection in a subject, as previously described herein. Other direct and indirect methods will be known to those of ordinary skill in the art and may be employed to assess the subject's response to treatment.

To elicit the desired response, treatment may be varied by increasing or decreasing the amount of a therapeutic composition, by changing the method of administration, by changing the dosage timing, by changing the pharmaceutical carrier, and so on. The effective amount will vary with the particular condition being treated, the age and physical condition of the subject being treated, the severity of the condition, the duration of the treatment, the nature of the concurrent therapy (if any), the specific route of administration, and the like factors within the knowledge and expertise of the health practitioner. For example, an effective amount may depend upon the degree to which a subject has colonization of streptococcal bacteria. For example, prophylactic treatment to prevent infection in an subjected suspected of exposure to streptococcal bacteria, in the presence of a positive or negative streptococcal culture, may comprise lower dosage and fewer administrations than treatment of a subject known to have streptococcal infection having a positive culture or test for streptococcal bacteria as described herein. In one embodiment, an effective amount will be in the range from 0.1 mg to 5000 mg hyaluronic acid, analog, or agent that binds the hyaluronic acid binding region of CD44, preferably from 1 mg to 500 mg.

It is generally preferred that a maximum dose of the hyaluronic acid, analog, or other agent that binds to the hyaluronic acid binding region of CD44 (alone or in combination with other therapeutic agents) be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The doses of hyaluronic acid, analog, or other agent that binds to the hyaluronic acid binding region of CD44 administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

Various modes of administration will be known to one of ordinary skill in the art which effectively deliver the hyaluronic acid, analog, or other agent that binds to the hyaluronic acid binding region of CD44 to a desired tissue, cell or bodily fluid. Administration may include but is not limited to: topical, intravenous, oral, intracavity, intrathecal, intrasynovial, buccal, sublingual, intranasal, transdermal, intravitreal, subcutaneous, intramuscular, and intradermal administration. Standard references in the art (e.g., Remington's Pharmaceutical Sciences, 18th edition, 1990) provide modes of administration and formulations for delivery of various pharmaceutical preparations and formulations in pharmaceutical carriers. The preferred method for administering the hyaluronic acid, analog, or other agent that binds to the hyaluronic acid binding region of CD44 is oral and most preferred is administration to the pharynx by swallowing. In other embodiments, the preferred method for administering the hyaluronic acid, analog, or other agent that binds to the hyaluronic acid binding region of CD44 is intranasal. Other protocols that are useful for the administration of hyaluronic acid, analog, or other agent that binds to the hyaluronic acid binding region of CD44 will be known to one of ordinary skill in the art, in which the dose amount, schedule of administration, sites of administration, mode of administration (e.g., intra-organ) and the like vary from those presented herein.

The pharmaceutical compositions used in the foregoing methods preferably are sterile, although this is not essential for oral or intranasal dosages, and contain an effective amount of hyaluronic acid, analog, or other agent that binds to the hyaluronic acid binding region of CD44 for producing the desired response in a unit of weight or volume suitable for administration to a patient.

Administration of hyaluronic acid, analog, or other agent that binds to the hyaluronic acid binding region of CD44 to mammals other than humans, e.g. for testing purposes or veterinary therapeutic purposes, is carried out under substantially the same conditions as described above. It will be understood by one of ordinary skill in the art that this invention is applicable to both human and animal diseases which can be treated by hyaluronic acid, analog, or other agent that binds to the hyaluronic acid binding region of CD44. Thus this invention is intended to be used in husbandry and veterinary medicine as well as in human therapeutics.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Hyaluronic acid, analog, or other agent that binds to the hyaluronic acid binding region of CD44 may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the hyaluronic acid, analog, or other agent that binds to the hyaluronic acid binding region of CD44, and with each other, in a manner such that there is no interaction which would substantially impair the desired pharmaceutical efficacy.

The pharmaceutical compositions may contain suitable buffering agents, as described above, including: acetate, phosphate, citrate, glycine, borate, carbonate, bicarbonate, hydroxide (and other bases) and pharmaceutically acceptable salts of the foregoing compounds.

The pharmaceutical compositions also may contain, optionally, suitable preservatives, such as: benzalkonium chloride; chlorobutanol; parabens, and thimerosal.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Preferably, pharmaceutical compositions are for oral or nasal administration. Pharmaceutical compositions for nasal administration may include: drops, suspensions, aerosols, inhalation sprays, and other nasal administration methods known to those of skill in the art. Compositions suitable for oral administration may be presented as discrete units, such as capsules, tablets, lozenges, chewing gum each containing a predetermined amount of the active compound. Preferred compositions include, but are not limited to: chewing gum, lozenges, and solutions or suspensions in aqueous liquids or non-aqueous liquids such as a syrup, elixir, or an emulsion. Preferred pharmaceutical compositions also include syrup such as a cough syrup, a solid solution (see U.S. Pat. No. 6,264,981), or a frozen solution (see U.S. Pat. No. 6,258,384).

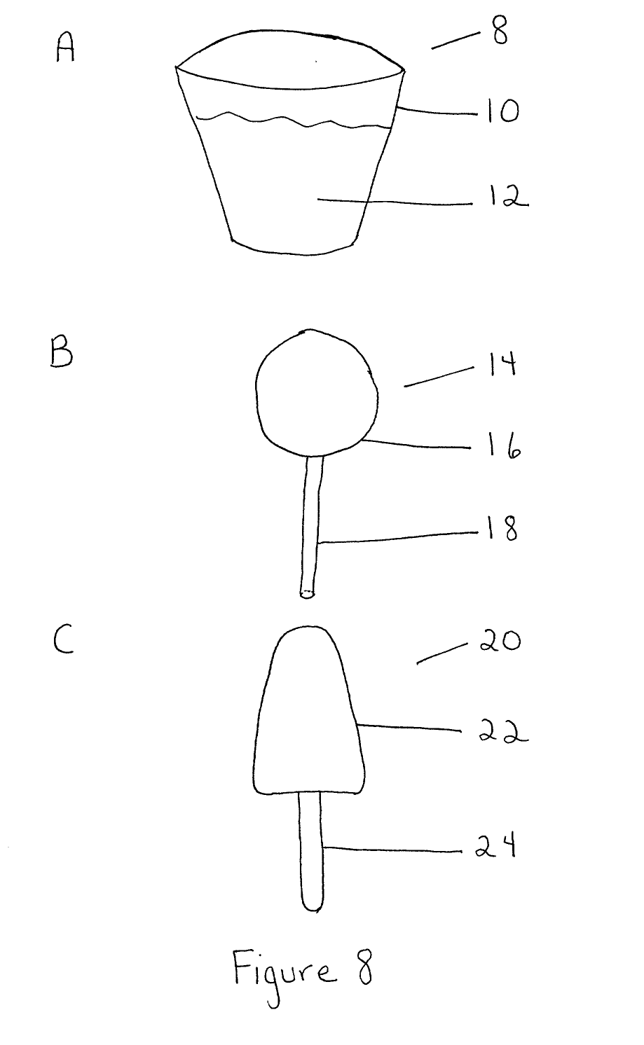

A syrup is thick liquid such as a mixture of sugar and water that is often flavored or medicated. An example of a syrup is cough syrup. A cough syrup is formulated typically with one or more of the following: dextromethorphan hydrobromide, pseudoephedrine hydrochloride, guaifenesin, acetaminophen, chlorpheniramine maleate, brompheniramine maleate, diphenhydramine hydrochloride, benzocaine, and dyclonine hydrochloride. Such ingredients may be distinguished from ingredients typically included in mouthwash and mouth rinse and toothpaste formulations, which include: thymol, eucalyptol, methyl salicylate, menthol, cetylpyridinium chloride and alcohol. Syrup ingredients may also be distinguished from ingredients typically included in mouth rinse and toothpaste formulations, which typically include sodium fluoride. A non-limiting example of a syrup medicinal product is given in FIG. 8A, in which 8 is the syrup medicinal product, 10 is the container, and 12 is the syrup.

A solid solution medicinal product is formulated typically with an aqueous base, dissolution agent, and pharmaceutical ingredient. A solid solution may also contain colorants, flavorants, solvents and co-solvents, coating agents, direct compression excipients, disintegrants, glidants, lubricants, opaquants, polishing agents, suspending agents, sweetening agents, anti-adherents, binders, and capsule diluents. The ingredients of a solid solution may also include preservatives, clarifying agents, emulsifying agents, antioxidants, levigating agents, plasticizers, surfactants, tonicity agents, and viscosity-increasing agents. The solid solution may be attached to a handle, an example of which, although not intended to be limiting, includes a stick or loop composed of wood, plastic, string, paper, metal, or other natural or synthetic material known to one of ordinary skill in the art. The solid solution may be on a platform that can be extended by the subject, for example, in the form of a push-up. It will be understood that the solid solution may be in any shape, and it may be of a size that can be placed in the mouth of the subject. In some embodiments the solid solution may be larger than can be placed in the mouth of the subject. A non-limiting example of a solid solution medicinal product is given in FIG. 8B, in which 14 is the solid solution medicinal product, 16 is the solid solution, and 18 is the handle.

A frozen solution medicinal product is formulated typically with active ingredients, flavoring agents, preservatives, food coloring, and a balance of water. The frozen solution medicinal product may be in a container, such as a cup or vessel, or a flexible sleeve, or it may be on a platform that can be extended by the subject, for example, in the form of a pushup. The frozen solution medicinal product may be attached to a handle, an example of which, although not intended to be limiting, includes a stick or loop composed of wood, plastic, string, paper, metal, or other natural or synthetic material known to one of ordinary skill in the art. The frozen solution medicinal product may be in any shape, and may be of a size that can be placed in the mouth of the subject. In some embodiments the frozen solution may be larger than can be placed in the mouth of the subject. A non-limiting example of a frozen solution medicinal product is given in FIG. 8C, in which 20 is the frozen solution medicinal product, 22 is the frozen solution, and 24 is the handle.

Compositions suitable for parenteral administration may be formulated according to known methods using suitable dispersing or wetting agents and suspending agents. The sterile injectable preparation also may be a sterile injectable solution or suspension in a nontoxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or diglycerides. In addition, fatty acids such as oleic acid may be used in the preparation of injectables. Carrier formulation suitable for oral, nasal, intrapulmonary, subcutaneous, intravenous, intramuscular, etc. administrations can be found in Remington 's Pharmaceutical Sciences, Mack Publishing Co., Easton, Pa., 18th ed. 1990.

### EXAMPLES

Methods

Bacterial Strains and Growth Conditions.

GAS strain B514-Sm is a spontaneous streptomycin-resistant derivative of B514/33, an M type 50 strain originally isolated from an epizootic infection of a mouse colony (Husmann, L. K., et al. 1996, Microb. Pathog. 20:213-24, Hook E. W., et al. 1960, Am. J. Hyg. 72:1111-119), UAB039 is an acapsular mutant of B514-Sm constructed by insertion of a nonreplicating plasmid within the hasA (hyaluronate synthase) gene (Husmann, L. K. et al. 1997, Infect. Immun. 65: 1422-1430). GAS strain 950771 is an M type 3 strain originally isolated from a patient with necrotizing fasciitis; strain 188 is an acapsular mutant of 950771 constructed by insertion of the ΩKm2 element, a kanamycin-resistance cassette flanked by transcriptional terminators, within the hasA gene (Ashbaugh, C. D., et al. 1998, J. Clin. Invest. 102:550-560). Bacteria were grown in liquid culture in Todd-Hewitt broth or on trypticase soy agar containing 5% sheep blood. GAS were grown in liquid culture to mid-exponential phase (A ₆₅₀=0.15), washed, and resuspended in serum-free keratinocyte basal medium without calcium or supplements (Clonetics, Inc., San Diego, Calif.) for in vitro attachment assays or in phosphate-buffered saline, pH 7.4, for intranasal inoculation of mice.

Mouse Strains.

Unless otherwise specified, experiments involving mice were performed using C57BL/6 mice (Jackson Laboratories, Bar Harbor, Me.). K5-CD44 transgenic mice with a selective deficiency in expression of CD44 in stratified squamous epithelia have been described previously (Kaya, G. et al. 1997, Genes and Development. 11:996-1007). These mice express a CD44-antisense transgene under the control of the keratin-5 promoter which targets expression of the antisense transgene to the basal cell compartment of stratified squamous epithelia.

Derivation and Characterization of Primary Mouse Keratinocytes.

Primary Mouse keratinocyte cultures were established from cells isolated from the epidermis of I to 3-day-old mice. Mouse skins were incubated overnight at 4° C. in 0.25% trypsin. The epidermis was separated from the dermis, minced and stirred to produce a single cell suspension before seeding onto collagen-coated tissue cultures wells (Nalge Nunc International, Naperville, Ill.). Cell cultures were incubated at 34° C. in 8% CO₂ for 3 to 10 days in serum-free keratinocyte basal medium without calcium, supplemented with KGM SingleQuots, original formula (Clonetics, Inc., San Diego, Calif.). CD44 expression on keratinocytes from each animal was assessed by immunofluorescence microscopy using mAb KM81 (Schrager, H. M. 1998, J. Clin. Invest. 101:1708-1716).

Bacterial Adherence Assays.

For bacterial adherence assays, keratinocytes were seeded at 10⁵ cells per well in collagen-coated tissue culture wells (Nalge Nunc International, Naperville, Ill.), incubated for 3 days at 34° C. in 8% CO₂, washed, and overlaid with medium containing 10⁶ CFU of GAS per well. After incubation for 1 hour at 34° C. in 8% CO₂ the keratinocyte monolayers were washed twice to remove nonadherent bacteria, then the number of cell-associated bacteria was determined by quantitative culture after releasing the keratinocytes with trypsin and lysing them in sterile water, experiments testing the effects of exogenous hyaluronic acid on GAS adherence were performed using the mouse keratinocyte cell line, PAM 2.12 (Song, I. S. 2000, Exp. Dermatol. 9:42-52). Cells were seeded in collagen-coated tissue culture wells at 10⁵ cells per well, cultured for 3 days at 37° C. in 5% CO₂, then inoculated with 10⁶ CFU of GAS per well in medium containing exogenous hyaluronic acid (from rooster comb, Sigma Chemicals, St. Louis, Mo.), no inhibitor, or a control polysaccharide, alginic acid, a high-molecular-weight polymer of mannuronic acid and guluronic acid (Protonal Durvillea Alginate, Pronova. Biopolymer, Inc., Portsmouth, N.H.). Cells were incubated for 45 minutes at 37° C. in 5% CO₂, then processed for enumeration of adherent bacteria as described above. In experiments to test whether exogenous hyaluronic acid could promote dissociation of GAS attached to keratinocytes, GAS were allowed to attach to keratinocytes in the absence of inhibitor; after 45 minutes, the medium was removed and replaced with fresh medium containing hyaluronic acid, no inhibitor, or alginic acid. After 45 minutes of incubation, the number of adherent bacteria was enumerated as described above.

Pharyngeal Colonization Studies in vivo.

For pharyngeal colonization experiments, 4 to 6 week-old female mice were anesthetized by inhalation of methoxyflurane, then inoculated intranasally with approximately 5×10⁶ CFU of GAS in 20 μl phosphate-buffered saline, pH 7.4. Throat swabs were collected from anesthetized mice daily and were plated on Todd-Hewitt-blood agar containing streptomycin 500 μg/ml to inhibit growth of normal flora. For some experiments, the bacterial inoculum was suspended in phosphate-buffered saline containing 50μg of mAb, either KM81, a rat anti-mouse CD44 mAb (Miyake, K. 1990, J. Exp. Med. 171:477-488), or 4H1, an isotype-matched control monoclonal antibody directed to Pseudomonas aeruginosa lipopolysaccharide (donated by Michael Preston, Brigham and Women's Hospital, Boston, Mass.). Both antibodies were purified by protein G affinity chromatography (GammaBind Plus Sepharose, Amersham Pharmacia Biotech, Piscataway, N.J.) prior to use. Examination by immunofluorescence microscopy of GAS cells incubated with mAb revealed no binding of either mAb to the organisms.

Immunohistochemistry.

Paraffin-embedded tissue sections through the mouse pharynx were deparaffinized as described previously (Schrager, H. M. et al. 1998, J. Clin. Invest. 101: 1708-1716). Sections were incubated with 0.1% hydrogen peroxide to quench endogenous peroxidase activity, then incubated for 1 hour with 1.5% normal rabbit serum, then with mAb KM81, 15 μg/ml for 30 minutes at room temperature. Slides were washed with phosphate-buffered saline, then incubated with biotin-conjugated rabbit anti-rat IgG, followed by horseradish peroxidase-streptavidin and the peroxidase substrate, diaminobenzidine tetrahydrochlorlde (Santa Cruz Biotechnology, Inc., Santa Cruz, Calif.). Slides were examined by light microscopy and photographed at 400× magnification under standard conditions.

Statistical Analysis.

Differences in attachment of GAS to keratinocytes were evaluated Using the Mann Whitney U test (Instat version 1.12, GraphPad Software, Inc. San Diego, Calif.). Repeated measures logistic regression was used to test for the effect of anti-CD44 mAb or erogenous hyaluronic acid treatment on the proportion of mice with positive throat cultures over three days of observation (Zeger, S. L. et al. 1986, Biometrics. 42:121-130).

### Example 1

GAS Attachment to Murine Keratinocytes in vitro is Mediated by Binding of the GAS Capsular Polysaccharide to CD44

To define the importance of GAS interaction with CD44 in pharyngeal colonization in vivo and to determine whether a murine model would be suitable for this investigation; experiments were preformed to establish that GAS attachment to primary cultured murine keratinocytes, like that to human keratinocytes, was mediated by binding of the GAS capsular polysaccharide to CD44.

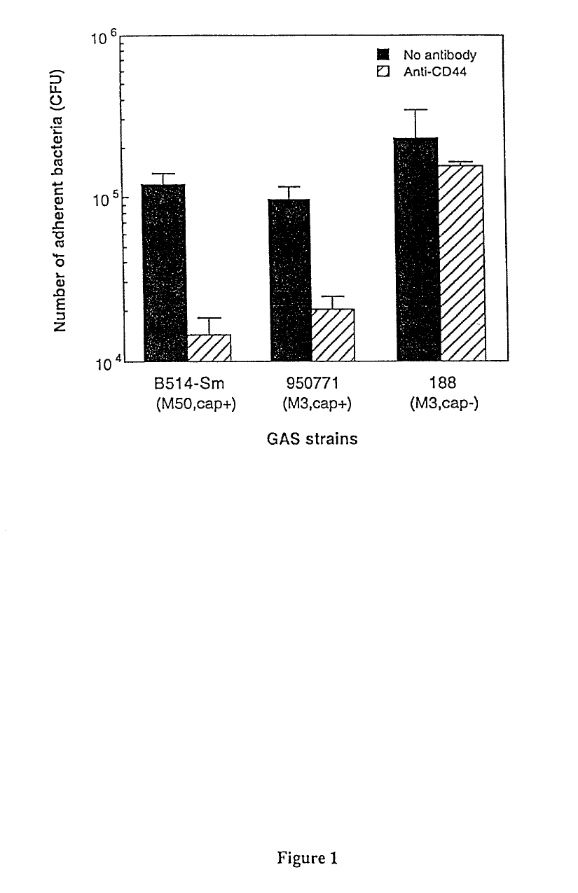

To examine the role of CD44 in GAS binding to murine keratinocytes, primary cultures were established of cells isolated from neonatal C57BL/6 mouse skin. Two GAS strains were studied: B514-Sm, an M-type 50 strain that has been shown to efficiently colonize the upper airway of mice after intranasal inoculation, and 950771, an M-type 3 isolate originally cultured from a patient with necrotizing fasciitis and typical of strains that cause human pharyngitis and invasive infection. Monoclonal antibody (mAb) KM81 directed to the hyaluronic acid binding site of mouse CD44 (Zheng, Z., et al. 1995, J. cell Biol. 130:485-495) inhibited by 75% binding of wild-type (i.e., encapsulated) GAS strains B514-Sm and 950771 to mouse keratinocytes (P<0.005 for both GAS strains compared to binding in the absence of mAb, FIG. 1). Mab KM81 had no significant effect on binding of strain 188, an isogenic acapsular mutant of strain 950771 derived from the wild-type GAS strain by inactivation of the hyaluronate synthase gene, hasA. The overall adherence of the capsule-deficient strain was equal to or greater than that of the encapsulated wild-type strain, but was mediated by binding interactions independent of CD44. These results are consistent with previous findings that the hyaluronic acid capsule masks alternative adhesins on the bacterial surface and prevents M-protein mediated attachment of GAS to human epithelial cells (Schrager, H. M. et al. 1998, J. Clin. Invest. 101:1708-1716, Courtney, H. S. et al. 1997, FEMS Microbiol. Let. 151:65-70).

### Example Two

### Encapsulated GAS Attach Poorly to CD44-deficient Keratinocytes from Transgenic Mice

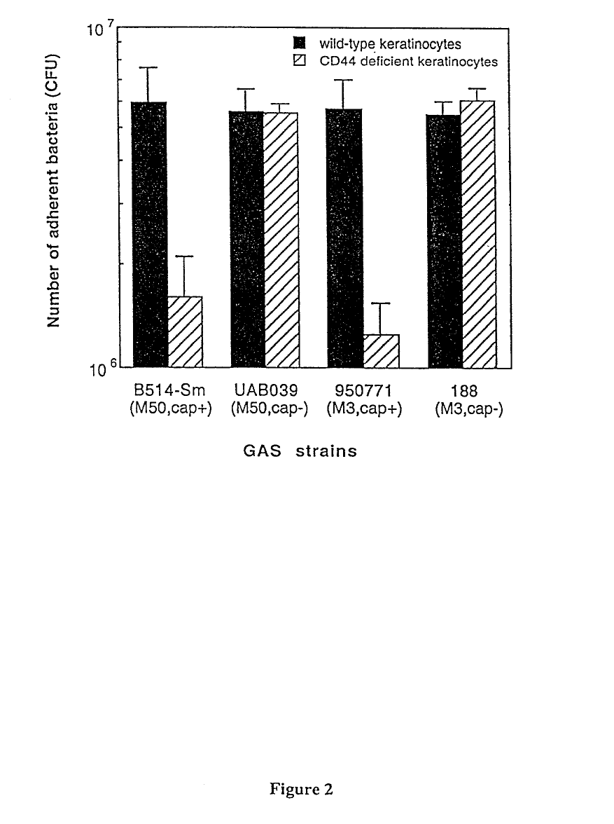

CD44-deficient transgenic mice were utilized to determine whether the selective loss of CD44 expression by keratinocytes affected GAS binding to keratinocytes in vitro and GAS colonization of the pharynx in vivo. K5-CD44 mice express a CD44-antisense transgene under the control of the keratin-5 promoter which targets expression of the antisense transgene to the basal cell compartment of stratified squamous epithelia (Kaya, G. 1997, Genes and Development. 111:996-1007). High level expression of the transgene results in complete loss of CD44 expression in all layers of the epidermis. Transgenic animals were screened for high-level expression of the antisense transgene by immunofluorescence microscopy of primary cultured keratinocytes using mAb KM81. The attachment of GAS to primary keratinocytes from wild-type C57BL/6 mice was compared to GAS attachment to K5-CD44 keratinocytes from antisense transgenic animals deficient in CD44 expression. In assays of GAS attachment in vitro, binding of wild-type GAS strains B514-Sm and 950771 to CD44-deficient keratinocytes was reduced by 73% and 78%, respectively, compared to binding to normal mouse keratinocytes (P<0.01 for binding of both GAS strains to wild-type versus CD44-deficient keratinocytes, FIG. 2). In contrast, binding of each of two isogenic GAS mutant strains lacking hyaluronic acid capsules to CD44-deficient keratinocytes was no different than that to normal keratinocytes. These results provide evidence that GAS attachment to mouse keratinocytes in vitro is mediated by binding of the GAS hyaluronic acid capsule to CD44.

### Example 3

### GAS Colonization of the Airway is Impaired in CD44-deficient Transgenic Mice in vivo

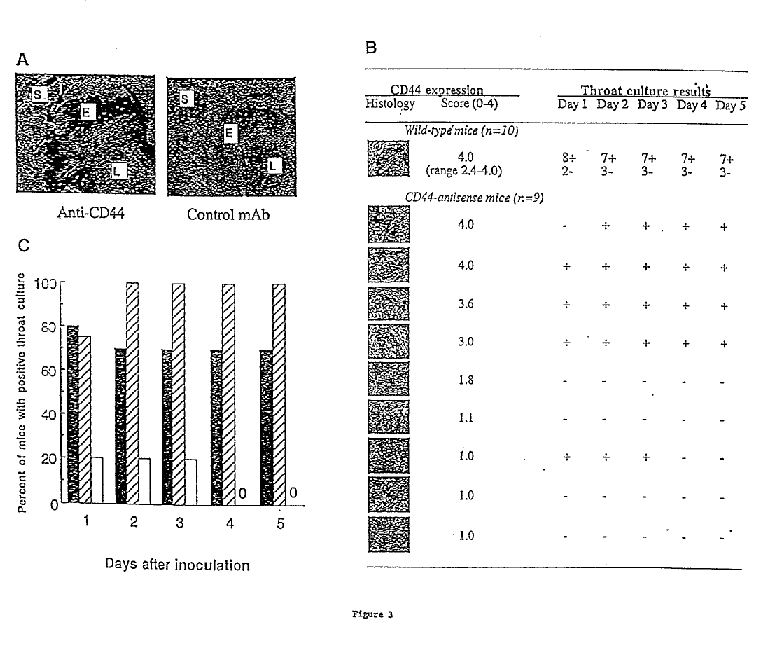

In vivo challenge experiments were performed to determine whether the observed in vitro interaction between the GAS capsule and CD44 reflected a role for CD44 as a receptor for GAS colonization of the pharyngeal epithelium in vivo. Groups of wild-type or K5-CD44 transgenic mice were inoculated intranasally with GAS strain B514-Sm. Throat cultures were obtained daily for 5 days after challenge to assess pharyngeal colonization. The degree of suppression of CD44 expression may be partial or complete in an individual animal depending on the extent of expression of the K5-CD44 antisense transgene. Therefore, the mice were euthanized at the end of the experiment and histologic sections of the pharynx were examined by microscopy after immunohistochemical staining to determine the degree of CD44 expression in the pharyngeal epithelium in individual animals. Four of 9 evaluable transgenic mice exhibited epithelial expression of CD44 similar to the levels in wild-type mice (histologic scores for CD44 expression of 3.0 to 4.0, compared to 2.4 to 4.0 for control mice). In 5 transgenic animals, epithelial CD44 expression was markedly reduced or undetectable (histologic scores of 1.0 to 1.8). Among the wild-type mice, throat cultures were positive for GAS in 8 to 10 animals 1 day after inoculation and in 7 of 10 animals with wild-type levels of CD44 expression: throat cultures were positive in 3 or 4 of 4 mice on each of 5 days after challenge. By contrast, among the 5 transgenic mice with low or absent epithelial CD44 expression, only a single animal had a positive throat culture on days 1, 2, and 3, and all 5 animals had negative cultures on days 4 and 5 (FIG. 3). Therefore, reduced CD44 expression on the pharyngeal epithelium was associated with rapid clearance of the GAS inoculum from the upper airway and failure of the bacteria to colonize the pharynx.

### Example 4

### Blocking GAS Binding to CD44 Prevents GAS Pharyngeal Colonization in Wild-type Mice

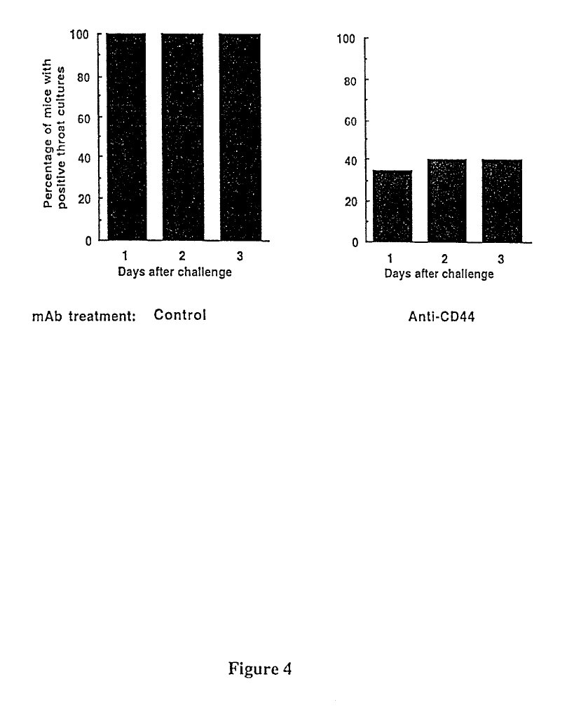

The experiments with transgenic mice described above indicated that pharyngeal expression of CD44 resulted in enhanced colonization by GAS. As an alternative means to characterize the role of CD44 as a GAS receptor, the effect of disrupting the interaction between the GAS capsule and CD44 was investigated in wild-type mice. For these studies, wild-type mice were inoculated intranasally with GAS strain B514-Sm mixed with either mAb to CD44 or an irrelevant control mAb directed to the lipopolysaccharide of Pseudomonas aeruginosa. Intranasal administration of anti-CD44 mAb KM81 together with the GAS challenge reduced colonization by ≧60% on each of 3 days after inoculation compared to coadministration of the control antibody with the bacterial inoculum (P<0.0001, FIG. 4). Thus, antibody specific for the hyaluronate-binding domain of the CD44 receptor blocked not only GAS attachment to epithelial cells in vitro, but also pharyngeal colonization by GAS in vivo.

### Example 5

### Exogenous Hyaluronic Acid Blocks GAS Binding to Murine Keratinocytes

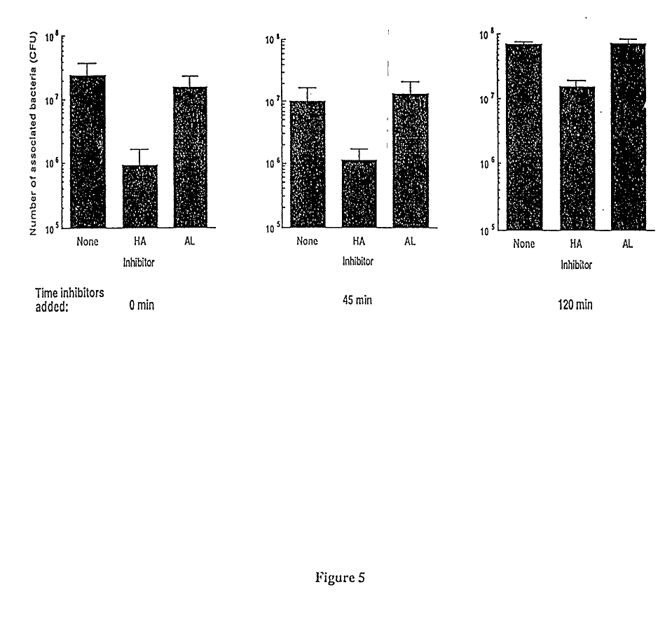

To investigate the specificity of the interaction between the GAS capsule and CD44 on epithelial cells, the ability of exogenous hyaluronic acid to compete with GAS for binding to murine keratinocytes was examined. Addition of exogenous hyaluronic acid inhibited GAS attachment to the murine keratinocyte cell line PAM2.12 by >95% (P<0.03, FIG. 5). Alginic acid, another high molecular weight acidic polysaccharide, had no effect, supporting the hypothesis that adherence depends on a specific interaction between the GAS hyaluronic acid capsule and CD44. Addition of exogenous hyaluronic acid up to 45 minutes after adding GAS to the keratinocytes still resulted in >90% reduction in bacterial attachment (P<0.03); if addition of hyaluronic acid was delayed for 2 hours, GAS attachment was reduced by 70-80% (P<0.03). These results suggested that the capsule-CD44 binding interaction may be important during the early phase of bacterial attachment, while alternative ligand-receptor interactions may supervene during the later phase.

### Example 6

### Pretreatment of Mice with Exogenous Hyaluronic Acid Reduces GAS Colonization of the Pharynx

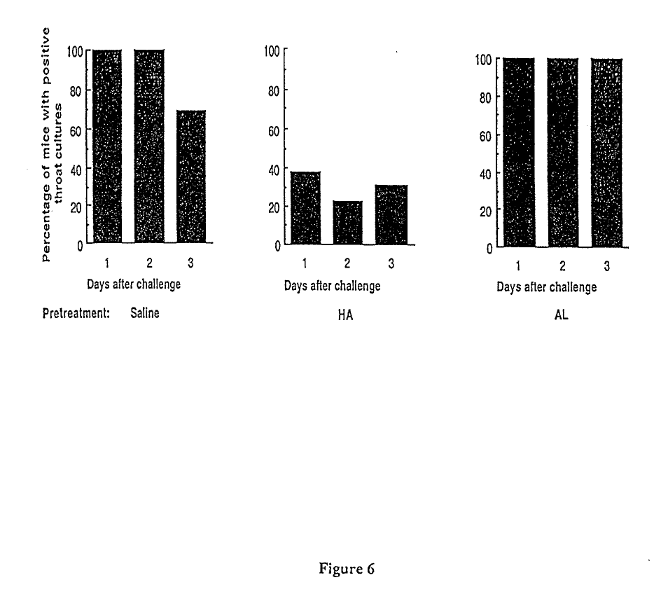

The experiments described above indicate that exogenous hyaluronic acid could compete for GAS binding to epithelial cells in vitro. Whether the same phenomenon might operate in vivo in the mouse model of pharyngeal colonization was examined. Mice were pretreated by intranasal administration of hyaluronic acid (20 μl of a 1 mg/ml solution) 5 hours prior to intranasal inoculation with GAS. Colonization of hyaluronic acid pretreated animals was reduced by approximately 60 to 80% compared to control animals pretreated with phosphate-buffered saline or animals pretreated with alginic acid (P<0.0001, FIG. 6). That hyaluronic acid pretreatment can reduce colonization upon bacterial challenge further supports the hypothesis that interaction with CD44 mediates GAS attachment to the pharyngeal mucosa. Furthermore, it suggests the possibility that topically administered hyaluronic acid or an analog that binds to CD44 might be an effective means of preventing GAS pharyngeal infection during a period of intensive exposure such as an outbreak or epidemic situation.

### Example 7

### Use of Hyaluronic Acid to Inhibit Binding to Human Epithelial Cells by multiple Bacterial Species

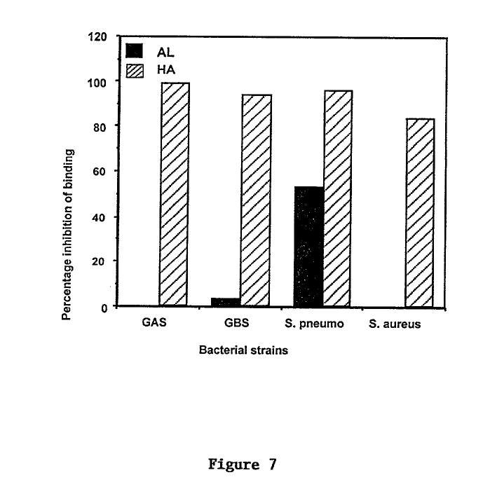

Keratinocyte monolayers were pre-incubated with alginate (AL, solid bars) or hyaluronic cid (HA, hatched bars) prior to surface inoculation with either Group A Streptococcus (GAS, strain 950771), Group B Streptococcus (GBS, strain 515), Streptococcus pneumoniae (S. pneumo, type 6), or Staphylococcus aureus (S. aureus, type 8) at a multiplicity of infection of 10 bacteria per keratinocyte for 1 hour. FIG. 7 illustrates that HA pre-treatment inhibited by more than 80% binding of each of the bacterial species tested. These data suggest that application of HA at mucosal (eg, throat or nose) sites can prevent infections due to both streptococcal and staphylococcal bacteria.

Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference.

What is claimed is:

1. A method for treating a subject to reduce the likelihood of streptococcal infection comprising
administering orally to a subject in need of such treatment an agent that binds to a hyaluronic acid-binding region of a CD44 protein in an amount effective to interfere with adhesion of streptococcal bacteria to CD44 protein in the subject and inhibit streptococcal colonization of the pharynx.

2. The method of claim 1, wherein the effective amount of the agent administered to the subject in need of such treatment statistically reduces the likelihood of infection.

3. The method of claim 1, wherein the agent is administered to a subject suspected of exposure to Group A streptococcus or Group C streptococcus.

4. The method of claim 3, wherein the subject is known to have been exposed to streptococcal bacteria.

5. The method of claim 3, wherein the subject is likely to be exposed to streptococcal bacteria.

6. The method of claim 1, wherein the subject is a human.

7. The method of claim 1, wherein the subject is in need of such treatment to reduce the likelihood of streptococcal pharyngitis.

8. The method of claim 1, wherein the CD44 protein in the subject in need of such treatment is located on the pharynx.

9. The method of claim 1, wherein the agent is hyaluronic acid or an analog of hyaluronic acid.

10. The method of claim 9, wherein the agent is a peptide.

11. The agent of claim 10, wherein the peptide is an antibody.

12. A method for treating a subject to reduce the likelihood of streptococcal infection comprising
administering orally to a subject in need of such treatment an agent that binds to a hyaluronic acid-binding region of a CD44 protein in an amount effective to displace streptococcal bacteria bound to CD44 protein in the subject and inhibit streptococcal colonization of the pharynx.

13. The method of claim 12, wherein the effective amount of the agent administered to the subject in need of such treatment statistically reduces the likelihood of infection.

14. The method of claim 12, wherein the agent is administered to a subject suspected of exposure to Group A streptococcus or Group C streptococcus.

15. The method of claim 14, wherein the subject has not been determined to have streptococcal infection.

16. The method of claim 14, wherein the subject has been determined to have streptococcal infection.

17. The method of claim 12, wherein the subject is a human.

18. The method of claim 12, wherein the subject is in need of such treatment to reduce the likelihood of streptococcal pharyngitis.

19. The method of claim 12, wherein the CD44 protein in the subject in need of such treatment is located on the pharynx.

20. The method of claim 12, wherein the agent is hyaluronic acid or an analog of hyaluronic acid.

21. The method of claim 12, wherein the agent is a peptide.

22. The agent of claim 21, wherein the peptide is an antibody.

23. A method for treating a subject to reduce the likelihood of streptococcal infection comprising
administering orally to a subject in need of such treatment an agent that binds to a hyaluronic acid-binding region of a CD44 protein of a mucosal membrane in an amount effective to interfere with adhesion of streptococcal or staphylococcal bacteria to the mucosal membrane in the subject.

24. The method of claim 23, wherein the hyaluronic acid interferes with adhesion of streptococcal bacteria to CD44 protein of a mucosal membrane in the subject.

25. The method of claim 23, wherein the effective amount of the agent administered to the subject in need of such treatment statistically reduces the likelihood of infection.

26. The method of claim 23, wherein the agent is administered to a subject suspected of exposure to Group A streptococcus, Group C streptococcus, Group B streptococcus, Streptococcus pneumoniae, or Staphylococcus aureus.

27. The method of claim 26, wherein the subject is known to have been exposed to streptococcal or staphylococcal bacteria.

28. The method of claim 26, wherein the subject is likely to be exposed to streptococcal or staphylococcal bacteria.

29. The method of claim 23, wherein the subject is a human.

30. The method of claim 23, wherein the subject is in need of such treatment to reduce the likelihood of streptococcal or staphylococcal pharyngitis.

31. The method of claim 23, wherein the CD44 protein of the mucosal membrane in the subject in need of such treatment is on the pharynx.

32. The method of claim 23, wherein the agent is hyaluronic acid or an analog of hyaluronic acid.

33. The method of claim 32, wherein the agent is a peptide.

34. The agent of claim 33, wherein the peptide is an antibody.

35. A method for treating a subject to reduce the likelihood of streptococcal or staphylococcal infection comprising administering orally to a subject in need of such treatment an agent that binds to a hyaluronic acid-binding region of a CD44 protein of a mucosal membrane in an amount effective to displace streptococcal or staphylococcal bacteria bound to the mucosal membrane in the subject.

36. The method of claim 35, wherein the hyaluronic acid interferes with adhesion of streptococcal bacteria to CD44 protein of a mucosal membrane in the subject.

37. The method of claim 35, wherein the effective amount of the agent administered to the subject in need of such treatment statistically reduces the likelihood of infection.

38. The method of claim 35, wherein the agent is administered to a subject suspected of exposure to Group A streptococcus, Group C Streptococcus, Group B streptococcus, Streptococcus pneumoniae, or Staphylococcus aureus.

39. The method of claim 38, wherein the subject has not been determined to have streptococcal or staphylococcal infection.

40. The method of claim 38, wherein the subject has been determined to have streptococcal or staphylococcal infection.

41. The method of claim 35, wherein the subject is a human.

42. The method of claim 35, wherein the subject is in need of such treatment to reduce the likelihood of streptococcal or staphylococcal pharyngitis.

43. The method of claim 35, wherein the CD44 protein of the mucosal membrane in the subject in need of such treatment is on the pharynx.

44. The method of claim 35, wherein the agent is hyaluronic acid or an analog of hyaluronic acid.

45. The method of claim 35, wherein the agent is a peptide.

46. The agent of claim 35, wherein the peptide is an antibody.

47. A method for treating a subject to reduce the likelihood of streptococcal or staphylococcal infection comprising
administering nasally to a subject in need of such treatment who does not have a nasal cavity wound, an agent that binds to a hyaluronic acid-binding region of a CD44 protein of a mucosal membrane in an amount effective to interfere with adhesion of streptococcal or staphylococcal bacteria to the mucosal membrane in the subject.

48. The method of claim 47, wherein the hyaluronic acid interferes with adhesion of streptococcal bacteria to CD44 protein of a mucosal membrane in the subject.

49. The method of claim 47, wherein the effective amount of the agent administered to the subject in need of such treatment statistically reduces the likelihood of infection.

50. The method of claim 47, wherein the agent is administered to a subject suspected of exposure to Group A streptococcus, Group C streptococcus, Group B streptococcus, Streptococcus pneumoniae, or Staphylococcus aureus.

51. The method of claim 50, wherein the subject is known to have been exposed to streptococcal or staphylococcal bacteria.

52. The method of claim 50, wherein the subject is likely to be exposed to streptococcal or staphylococcal bacteria.

53. The method of claim 47, wherein the subject is a human.

54. The method of claim 47, wherein the subject is in need of such treatment to reduce the likelihood of streptococcal or staphylococcal pharyngitis.

55. The method of claim 47, wherein the CD44 protein of the mucosal membrane in the subject in need of such treatment is on the pharynx.

56. The method of claim 47, wherein the agent is hyaluronic acid or an analog of hyaluronic acid.

57. The method of claim 56, wherein the agent is a peptide.

58. The agent of claim 57, wherein the peptide is an antibody.

59. The method of claim 47, wherein the CD44 protein of the mucosal membrane in the subject in need of such treatment is on a nasal mucosal membrane.

60. A method for treating a subject to reduce the likelihood of streptococcal or staphylococcal infection comprising
administering nasally to a subject in need of such treatment who does not have a nasal cavity wound, an agent that binds to a hyaluronic acid-binding region of a CD44 protein of a mucosal membrane in an amount effective to displace streptococcal or staphylococcal bacteria bound to the mucosal membrane in the subject.

61. The method of claim 60, wherein the hyaluronic acid interferes with adhesion of streptococcal bacteria to CD44 protein of a mucosal membrane in the subject.

62. The method of claim 60, wherein the effective amount of the agent administered to the subject in need of such treatment statistically reduces the likelihood of infection.

63. The method of claim 60, wherein the agent is administered to a subject suspected of exposure to Group A streptococcus, Group C streptococcus, Group B streptococcus, Streptococcus pneumoniae, or Staphylococcus aureus.

64. The method of claim 63, wherein the subject has not been determined to have streptococcal or staphylococcal infection.

65. The method of claim 63, wherein the subject has been determined to have streptococcal or staphylococcal infection.

66. The method of claim 60, wherein the subject is a human.

67. The method of claim 60, wherein the subject is in need of such treatment to reduce the likelihood of streptococcal or staphylococcal pharyngitis.

68. The method of claim 60, wherein the CD44 protein of the mucosal membrane in the subject in need of such treatment is on the pharynx.

69. The method of claim 60, wherein the agent is hyaluronic acid or an analog of hyaluronic acid.

70. The method of claim 60, wherein the agent is a peptide.

71. The agent of claim 70, wherein the peptide is an antibody.

72. The method of claim 60, wherein the CD44 protein of the mucosal membrane in the subject in need of such treatment is on a nasal mucosal membrane.

73. A medicinal product comprising a syrup containing an amount of hyaluronic acid effective to interfere with adhesion of streptococcal bacteria to CD44 protein in a subject and to inhibit streptococcal colonization of a pharynx of the subject.

74. A medicinal product comprising a syrup containing an amount of hyaluronic acid effective to displace streptococcal bacteria bound to CD44 protein in a subject and to inhibit streptococcal colonization of a pharynx of the subject.

75. A medicinal product comprising a syrup containing an amount of hyaluronic acid effective to interfere with adhesion of streptococcal or staphylococcal bacteria to a mucosal membrane in a subject.

76. The medicinal product of claim 75, wherein the mucosal membrane in the subject is a pharynx of the subject.

77. A medicinal product comprising a syrup containing an amount of hyaluronic acid effective to displace streptococcal or staphylococcal bacteria bound to a mucosal membrane in a subject.

78. The medicinal product of claim 77, wherein the mucosal membrane in the subject is a pharynx of the subject.

79. A medicinal product comprising a frozen solution containing an amount of hyaluronic acid effective to interfere with adhesion of streptococcal bacteria to CD44 protein in a subject and to inhibit streptococcal colonization of a pharynx of the subject.

80. A medicinal product comprising a frozen solution containing an amount of hyaluronic acid effective to displace streptococcal bacteria bound to CD44 protein in a subject and to inhibit streptococcal colonization of a pharynx in the subject.

81. A medicinal product comprising a frozen solution containing an amount of hyaluronic acid effective to interfere with adhesion of streptococcal or staphylococcal bacteria to a mucosal membrane in a subject.

82. The medicinal product of claim 81, wherein the mucosal membrane in the subject is a pharynx of the subject.

83. A medicinal product comprising a frozen solution containing an amount of hyaluronic acid effective to displace streptococcal or staphylococcal bacteria bound to a mucosal membrane in a subject.

84. The medicinal product of claim 83, wherein the mucosal membrane in the subject is a pharynx of the subject.

85. A medicinal product comprising a solid solution containing an amount of hyaluronic acid effective to interfere with adhesion of streptococcal bacteria to CD44 protein in a subject and to inhibit streptococcal colonization of a pharynx of the subject.

86. A medicinal product comprising a solid solution containing an amount of hyaluronic acid effective to displace streptococcal bacteria bound to CD44 protein in a subject and to inhibit streptococcal colonization of a pharynx of the subject.

87. A medicinal product comprising a solid solution containing an amount of hyaluronic acid effective to interfere with adhesion of streptococcal or staphylococcal bacteria to a mucosal membrane in a subject.

88. The medicinal product of claim 87, wherein the mucosal membrane in the subject is a pharynx of the subject.

89. A medicinal product comprising a solid solution containing an amount of hyaluronic acid effective to displace streptococcal or staphylococcal bacteria bound to a mucosal membrane in the subject.

90. The medicinal product of claim 89, wherein the mucosal membrane in the subject is a pharynx of the subject.
